(19) United States
(12) Patent Application Publication
(10) Pub. No.: US 2025/0387410 A1
(43) Pub. Date: Dec. 25, 2025

(54) TREATMENT OF ANTI-VEGF REFRACTORY OPHTHALMIC RETINAL EYE CONDITIONS

(71) Applicant: Eye Co Pty Ltd., New South Wales (AU)

(72) Inventor: Phillip Leslie Penfold, Moruya, South Wales (AU)

(21) Appl. No.: 18/837,905

(22) PCT Filed: Feb. 15, 2023
(86) PCT No.: PCT/AU2023/050103

§ 371 Date: Aug. 13, 2024

(30) Foreign Application Priority Data
Feb. 15, 2022 (AU) 2022900318

(51) Int. Cl. A61K 31/57; A61K 9/00; A61K 47/46; A61P 27/02

(52) U.S. Cl. CPC A61K 31/57; A61K 9/0019; A61K 47/46; A61P 27/02

(57) ABSTRACT

A method for treating a subject having a retinal eye condition that is refractory to anti-VEGF treatments is disclosed. The method comprises administering a therapeutically effective amount of one or more steroids subsequent to anti-VEGF treatment, thereby treating the retinal eye condition. Also disclosed is a method for treating a subject having a retinal eye condition that is refractory to anti-VEGF treatments, the method comprising a therapeutically effective amount of one or more compounds capable of modulating an activity of a steroid receptor, subsequent to anti-VEGF treatment. Further disclosed is a method for treating a subject having a retinal eye condition that is refractory to anti-VEGF treatments comprising administering a therapeutically acceptable formulation of a steroid, and at least a second therapeutically active compound in a concentration and dose sufficient to ameliorate the retinal eye condition, subsequent to anti-VEGF treatment.

### FIELD OF THE INVENTION

The present invention relates to the field of the treatment of one or more ophthalmic conditions using one or more therapeutic compounds when one or more other forms of treatment have not been effective; have ceased to be effective; or have reduced efficacy.

In one form, the invention relates to administration of one or more therapeutic compounds to treat one or more retinal eye conditions that is refractory to one or more other therapeutics.

In one particular aspect, the present invention is suitable for use in the treatment of one or more retinal eye conditions that are refractory to one or more anti-VEGF treatments.

It will be convenient to hereinafter describe the invention in relation to age-related macular degeneration (AMD) and diabetic macular edema (DME), however it should be appreciated that the present invention is not limited to these disorders only and relates to a wide range of conditions that affect the retina of the eye, and which are routinely treated with anti-VEGF agents.

### BACKGROUND ART

It is to be appreciated that any discussion of documents, devices, acts or knowledge in this specification is included to explain the context of the present invention. Further, the discussion throughout this specification comes about due to the realisation of the inventor and/or the identification of certain related art problems by the inventor. Moreover, any discussion of material such as documents, devices, acts or knowledge in this specification is included to explain the context of the invention in terms of the inventor's knowledge and experience and, accordingly, any such discussion should not be taken as an admission that any of the material forms part of the prior art base or the common general knowledge in the relevant art in Australia, or elsewhere, on or before the priority date of the disclosure and claims herein.

Vascular endothelial growth factor (VEGF) is a signal protein produced by cells that stimulate formation of new blood vessels in the embryonic circulatory system (vasculogenesis), and growth of blood vessels from pre-existing vasculature (angiogenesis), such as after injury.

VEGF exerts biological effects through the interaction with transmembrane receptors such as tyrosine kinase receptors VEGFR1 and VEGFR2. The ligands which specifically bind to VEGFR1 are VEGF-A, —B and PlGF while those bind to VEGFR2 are VEGF-A, -C, -D and -E.

However, over expression of VEGF can cause vascular disease, including in the retina of the eye. Excessive production of VEGF causes retinal damage by:
increasing permeability of existing blood vessels, so that they leak fluid and proteins into the retina causing oedema in surrounding tissue; and
stimulating growth of new blood vessels, within the retina, and outward from the retina onto the surface of the vitreous, and also into the space between the retina and the choroid, usually just under the retinal pigment epithelium.

Anti-VEGF therapeutics block VEGF. They can slow the growth of blood vessels in the eye and are extensively used for treatment for certain retinal diseases such as:
Macular degeneration (MD), particularly neovascular age-related macular degeneration (wet AMD) involving abnormal overgrowth of blood vessels from the choroid into the retina which can leak exudate and fluid;
swelling of the retina, known as macular edema (ME), including angio-graphic cystoid macular edema, diabetic macular edema (DME) and cystoid macular edema (CME) typically caused by disease (eg diabetes), injury or eye surgery;
diabetic retinopathy (DR), in which abnormal new blood vessels form at the back of the eye as part of proliferative diabetic retinopathy (PDR) which can burst and bleed; and
retinal vein occlusion, such as occlusion of the central retinal vein (CRVO) or branch retinal vein (BRVO), usually due to thrombosis.

For example, the first-line anti-VEGF treatment for neovascular AMD consists of intravitreal injection of humanised monoclonal antibodies, Bevacizumab (Avastin™) and Ranibizumab (Lucentis™) and recombinant fusion protein Aflibercept (Eylea™).

In recent times combination therapies have started to be investigated, with the aim of dual-target inhibition of VEGR-A and VEGR-C/-D. An example of a potential treatment of this type is OPT-302 (sVEGFR-3) which is a ‘trap’ inhibitor of VEGF-C and VEGF-D being developed by Opthea Limited for use in combination with any of the existing anti-VEGF-A agents, biosimilars or novel therapies in development for wet AMD and DME.

Various studies have been conducted to investigate a potential link between Macular Atrophy (MA) of the retinal pigment epithelium (RPE) in patients with AMD and anti-VEGF treatment. A review article to Horani et al, which considered various clinical studies into MA noted that “Taking figures and results from all relevant studies into consideration, while expressing the percentages out of the total number of study eyes that have been assessed up till the mean follow-up duration, the mean percentage of eyes with no baseline MA was 80%, with a median of 89%. The mean percentage of MA incidence is 29%, which was also the median. The mean prevalence of MA by the end of the study was 50%, the median was 46%. For the studies included the number of studied eyes that were treated and continuously followed up till the end of the mean follow-up period ranged between 28 and 1024 eyes, with a median of 118 eyes, and a mean cohort size of 242 eyes.” (Mania Horani, Sajjad Mahmood, Tariq M. Aslam (2020) A Review of Macular Atrophy of the Retinal Pigment Epithelium in Patients with Neovascular Age-Related Macular Degeneration: What is the Link? Part II. Ophthalmology and Therapy 9:35-75).

### Geographic Atrophy (GA)

GA is an eye disease that can lead to significant vision loss and affects approximately five million people around the world.

An analysis of the American Academy of Ophthalmology (AAO) IRIS® (Intelligent Research in Sight) Registry clinical data has been reported to show significant disease progression over a two-year period in more than 69,000 patients with GA, highlighting the urgent need for treatment (C. Francois and E. Rahimy (2020) New Findings from the IRIS Registry: Evaluating Geographic Atrophy in Real-world Clinical Practice presented at American Academy of Ophthalmology (AAO) 2020 Conference).

It was also reported that patients were nearly three times more likely to develop new onset wet age-related macular degeneration (AMD) in an eye with GA when wet AMD had already been detected in the contralateral eye (ibid).

Progression from GA to new onset wet AMD was reported to be observed in 4.7% of patients with bilateral GA (GA in both eyes) and 13.3% of patients with wet AMD in the contralateral eye during the first 12 months, with the rate at twenty-four months 8.2% and 21.6% in bilateral GA and wet AMD in the contralateral eye, respectively (ibid).

### Neovascular AMD

Anti-VEGF drugs have had a significant effect as a treatment for eye disease. However, some subjects are non-responsive, or have a poor response to anti-VEGF agents, or have a slow loss of efficacy of anti-VEGF agents after repeated administration over time.

For example, neovascular AMD patients are treated with anti-VEGF agents until completely ‘dry’, that is, there is no fluid in the sub-reginal space. If fluid returns, or vision deteriorates, treatment is resumed with the same anti-VEGF agent, but the condition may not respond. Some patients on anti-VEGF treatment start to become dry, but then start accumulating fluid again.

Intra-vitreal injections of anti-VEGF for AMD cannot be used ad infinitum for conditions such as AMD because they stop all vascular growth. The prime risk for macular degeneration is vascular insufficiency, and long-term use of anti-VEGF treatments can generate more vascular insufficiency and further application of anti-VEGF agents will lead to further risk of insufficient vascular supply.

Outer-retinal atrophy (cRORA) is atrophy of the RPE that can be related to an extended-period of anti-VEGF treatment for AMD. Clinically this presents as bare spots in the fundus, identifiable by methods such as Cirrus spectral domain optical coherence tomography (SD-OCT) imaging. The number of anti-VEGF injections has been shown to inversely correlate with cRORA area and growth. (Complete RPE and Outer Retinal Atrophy in Patients Receiving anti-VEGF Treatment for Neovascular Age-related Macular Degeneration, Eng et al, PLoS One 2020; 15(5)I e0232353, 5 May 2020)

### Diabetic Retinopathy

Macular degeneration occurs primarily through damage to the outer-retinal vasculature, the choroidal vasculature, whereas diabetic retinopathy occurs primarily through damage to intra-retinal vessels. In the proliferative form of diabetic retinopathy, VEGF is thought to drive the process of vascular proliferation.

Anti-VEGF agents are an emerging treatment for PDR, (and potentially non-proliferative DR) and may initially cause regression of PDR by reducing terminal neovascularisation due to ischemia. Intravitreal anti-VEGF agents may also be associated with increased fibrosis and regression of the vascular component of fibrovascular proliferation, leading to retinal detachment. (A. Fung & M. Hui, PDR: A New Anti-VEGF Era?, Miophthalmology, 10 Jul. 2018)

Accordingly, there is an ongoing need for alternative treatments for conditions affecting the retina of the eye.

### SUMMARY OF INVENTION

An object of the present invention is to provide an alternative therapy for retinal conditions when existing treatments do not have desired outcomes or cease to provide desired outcomes.

A further object of the present invention is to alleviate at least one disadvantage associated with the related art.

It is an object of the embodiments described herein to overcome or alleviate at least one of the above noted drawbacks of related art systems or to at least provide a useful alternative to related art systems.

In a first aspect of embodiments described herein there is provided a method for treating a subject having a retinal eye condition that is refractory to anti-VEGF treatments, the method comprising administering to the subject a therapeutically effective amount of one or more steroids, preferably one or more mineralocorticoids or glucocorticoids, subsequent to anti-VEGF treatment, thereby treating the retinal eye condition.

In a second aspect of embodiments described herein there is provided a method for treating a subject having a retinal eye condition that is refractory to anti-VEGF treatments, the method comprising administering to the subject a therapeutically effective amount of one or more compounds capable of modulating an activity of a steroid receptor, preferably a glucocorticoid receptor and/or mineralocorticoid receptor, subsequent to anti-VEGF treatment, thereby treating the retinal eye condition.

In a third aspect of embodiments described herein there is provided a method for treating a subject having a retinal eye condition that is refractory to anti-VEGF treatments, the method comprising administering to the individual; (a) a therapeutically acceptable formulation of a steroid suitable for delivery to the eye, and (b) at least a second therapeutically active compound in a concentration and dose sufficient to ameliorate the retinal eye condition, subsequent to anti-VEGF treatment.

In a particularly preferred embodiment of any one of the above aspects of the present invention, the steroid is one or more mineralocorticoid or glucocorticoid or a therapeutically active analogue, derivative, homolog, pharmaceutically acceptable salt or conjugate thereof.

The one or more mineralocorticoid or a therapeutically active analogue, derivative, homolog, pharmaceutically acceptable salt or conjugate thereof may comprise one or more of: 11-desoxycortisone (11-DC); fludrocortisone; fludrocortisone acetate (FA); fludrocortisone acetonide; Deoxycorticosterone acetate (DA); Deoxycorticosterone (DS); or Aldosterone; or a therapeutically active analogue, derivative, homolog, pharmaceutically acceptable salt or conjugate thereof.

The one or more glucocorticoid or a therapeutically active analogue, derivative, homolog, pharmaceutically acceptable salt or conjugate thereof may comprise one or more of: cortisol, cortisone, prednisone, prednisolone, methylprednisolone, dexamethasone, betamethasone, triamcinolone, triamcinolone acetonide, beclomethasone, fluocinolone or a therapeutically active analogue, derivative, homolog, pharmaceutically acceptable salt or conjugate thereof.

The one or more mineralocorticoid and/or more glucocorticoid or a therapeutically active analogue, derivative, homolog, pharmaceutically acceptable salt or conjugate thereof may comprise one or more dual action compounds, wherein each dual action compound is capable of modulating the activity of both a mineralocorticoid receptor and a glucocorticoid receptor.

The dual action compound may comprise one or more of triamcinolone; triamcinolone acetonide; cortisol; cortisone; prednisone; prednisolone; methylprednisolone; fludrocortisone; fludrocortisone acetate; fludrocortisone acetonide; or a therapeutically active analogue, derivative, homolog, pharmaceutically acceptable salt or conjugate thereof.

In a particularly preferred embodiment the one or more mineralocorticoid or one or more glucocorticoid or a therapeutically active analogue, derivative, homolog, pharmaceutically acceptable salt or conjugate thereof comprises fludrocortisone or a therapeutically active analogue, derivative, homolog, pharmaceutically acceptable salt or conjugate thereof. The therapeutically active analogue, derivative, homolog, pharmaceutically acceptable salt or conjugate thereof may comprise one or more of fludrocortisone acetate and fludrocortisone acetonide.

In one particular embodiment, the one or more mineralocorticoid and/or one or more glucocorticoid or a therapeutically active analogue, derivative, homolog, pharmaceutically acceptable salt or conjugate thereof comprises triamcinolone acetonide or a therapeutically active analogue, derivative, homolog, pharmaceutically acceptable salt or conjugate thereof.

In a particularly preferred embodiment any one of the above aspects of the present invention, the retinal eye condition is macular edema (ME) such as diabetic macular edema (DME), or age related macular degeneration (AMD) including wet-AMD or dry AMD.

Where used herein the term “refractory”, when used in relation to a retinal eye condition, is intended to refer to no response, poor response, adverse response, or loss of response over time to anti-VEGF therapy. The eye condition may become refractory at any time during the course of anti-VEGF therapy and may fail from the beginning or have an initial successful treatment period before becoming less effective, or even deleterious to the subject. The eye condition may become refractory due to many factors including sustained activation of other pathogenic pathways, tachyphylaxis, pharmacodynamic tolerance, changes to the neovascular architecture, redundant or compensatory angiogenic factors, sustained activation of complement system and inflammatory response, and genetic factors.

Where used herein the term “eye condition” includes any eye condition such as, early or sub-clinical stages of an eye disease which has proved refractory to anti-VEGR treatment.

According to any one of the above aspects, said retinal eye condition may be: an exudative eye condition, a back of the eye condition, macular degeneration including age-related macular degeneration (AMD) including both the dry (geographic atrophy) and wet (choroidal neovascularisation (CNV)), macula edema (ME) including diabetic macular edema (DME), angio-graphic cystoid macular edema, cistoid macular edema (CMO), diabetic retinopathy, (DR) including proliferative diabetic retinopathy (PDR) and retinal vein occlusion including central retinal vein occlusion (CRVO) or branch retinal vein occlusion (BRVO) maculopathy including an age related maculopathy (ARM), an exudative eye disease or condition, retinal pigment epithelium detachments (PED), forms of age related macular degeneration, a diabetic eye disease or condition including a diabetic retinopathy, corneal neovascularisation, cyclitis, Hippel-Lindel disease, retinopathy of prematurity (also known as retrolental fibroplasia), pterygium, histoplasmosis, iris neovascularisation, glaucoma, glaucoma-associated neovascularisation, Purtcher's retinopathy, ocular hypertension, macular oedema, Coats' disease, uveitis including anterior uveitis, Sicca syndrome, hereditary diseases associated with increased extra-intracellular lipid storage/accumulation, juvenile macular degeneration, an ocular allergy and an ocular tumour. The ocular tumour may comprise a retinoblastoma and/or a melanoma.

The eye disease or condition may comprise a back of eye disease or condition, including an exudative back of eye exudative disease or condition. The back of eye disease or condition may comprise an eye disease or condition involving the retina, macular and/or fovea in the posterior region of the eye. Examples of back of eye diseases include macular oedema, such as clinical macular oedema or angiographic cystoid macular oedema arising from various aetiologies, such as diabetes, exudative macular degeneration and macula oedema arising from laser treatment of the retina, retinal ischemia and choroidal neovascularisation, a retinal disease, an inflammatory disease, uveitis associated with neoplasms, such as retinoblastoma or psuedoglioma, neovascularisation following vitrectomy, a vascular disease and neovascularisation of the optic nerve. The retinal disease may be one or more of diabetic retinopathy, diabetic retinal oedema, retinal detachment, senile macular degeneration due to sub-retinal neovascularisation and myopic retinopathy. The vascular disease may be one or more of retinal ischemia, choroidal vascular insufficiency, choroidal thrombosis and neovascular retinopathies resulting from carotid artery ischemia.

In one embodiment of any one of the above forms, the eye disease or condition comprises dry AMD. Dry AMD may comprise early AMD and geographic atrophy (GA), distinct from exudative AMD.

The invention may find application to an exudative eye disease and/or condition, a back of the eye exudative eye disease and/or condition, age-related macular degeneration, wet age related macular degeneration, a diabetic macular oedema (DME), cystoid macular oedema (CMO); maculopathy; and/or an ocular tumour. The ocular tumour may comprise a retinoblastoma and/or a melanoma. The eye disease and/or condition may be a diabetic eye disease and/or condition. Other eye disease and/conditions include (non-infectious) conjunctivitis, anterior uveitis and an ocular allergy.

According to any one of the above aspects, said eye disease and/or condition may be a diabetic eye disease and/or condition.

An effective quantity of the compound of interest is preferably employed in the method of the invention. For formulations, the concentration of the therapeutic compound may be in the range of about 0.01 wt % to about 10 wt %. Typically, the concentration is in the range of about 0.025 wt % to about 2.5 wt %.

The phrase “therapeutically effective amount” is used herein to refer to an amount of therapeutic compound either solus or in combination with one or more other compounds that is sufficient to induce a therapeutic effect on the one or more retinal eye conditions. This phrase should not be understood to mean that the administration must completely eradicate the retinal eye condition. What constitutes a therapeutically effective amount will vary depending on condition, inter alia, the biopharmacological properties of the compound used, the retinal eye condition being treated, the frequency of administration, the mode of delivery, characteristics of the subject to be treated, the severity of the retinal eye condition and the response of the subject. These are the types of factors that the person skilled in the art will be aware of and will be able to account for when formulating compositions for a treatment as herein described.

The present invention may be used for medical or veterinary applications. The “subject” of treatment according to the present invention is a vertebrate animal, preferably a human.

In one embodiment of any one of the above aspects, the one or more steroids are injected into the eye. The injection may comprise suprachoroidal injection.

In another embodiment of any one of the above aspects, the one or more steroids are provided in a unit-dose formulation. The unit dose formulation may be provided in a pre-filled syringe. The pre-filled syringe may comprise two barrels. A first barrel may comprise the one or more steroids. A second barrel, different to the first barrel, may comprise one or more additional agent.

In another embodiment of any one of the above aspects, one or more pharmaceutically acceptable carriers, diluents or excipients may be comprised such as, one or more surfactant or wetting agent. The surfactant may comprise a polysorbate. The polysorbate may comprise one or more of polysorbate 20 and polysorbate 80. In a particular embodiment the surfactant comprises polysorbate 80. The pharmaceutically acceptable carrier, diluent or excipient may comprise carboxy methyl cellulose (CMC).

In one embodiment of any one of the above aspects, the one or more steroids further comprises one or more of a pH adjustment composition and water for injection. The pH adjustment composition may comprise hydrochloric acid and/or sodium hydroxide.

In another embodiment of any one of the above aspects, the one or more steroids comprises a pH from 6 to 8. The pH may comprise from 6 to 7.5.

In one embodiment of any one of the above aspects, the one or more mineralocorticoid and/or one or more glucocorticoid is comprised in a balanced salt solution. The balanced salt solution may comprise a saline and a buffer. The balanced salt solution comprises one or more of sodium chloride; potassium chloride; calcium chloride (dehydrate); magnesium chloride (hexahydrate); sodium acetate (trihydrate); sodium citrate (dehydrate); hydrochloric acid; sodium hydroxide and water for injection.

According to any one of the above aspects, the one or more steroids may comprise a sustained release composition.

In a particular embodiment of any one of the above aspects, the one or more steroids may be sterilized.

In one embodiment of any one of the above aspects, at least one additional agent may be administered.

According to any one of the above forms the one or more pharmaceutically acceptable carrier may comprise hemp, hemp oil or a pharmaceutically effective hemp or hemp oil extract. The hemp, hemp oil or pharmaceutically effective hemp or hemp oil extract may comprise a cannabinoid. The cannabinoid may comprise cannabidiol. The hemp, hemp oil or pharmaceutically active hemp or hemp oil extract may comprise a low Tetrahydrocannabinol (THC) hemp, hemp oil or pharmaceutically effective extract thereof. The hemp, hemp oil or pharmaceutically effective hemp or hemp oil extract may be obtained from a Cannabis ruderalis. The hemp, hemp oil or a pharmaceutically effective hemp or hemp oil extract may comprise a water-soluble dosage form. The hemp oil may be obtained from hemp seeds. The hemp oil may be cold-pressed.

According to one embodiment of any one of the above aspects, the hemp oil may comprise about 80% to 90% balanced Omega fatty acids. That is, hemp oil comprises Omega 3, (ALA), Omega 6 (LA), Omega 6 (GLA), and Omega 9 (oleic acid), which in combination may amount to 80% to 90% of the composition of the hemp oil.

In another embodiment of any one of the above aspects, the hemp oil may comprise about 88% balanced Omega fatty acids. That is, the hemp oil may comprise about 88 g Omega fatty acids per 100 g of hemp oil.

In yet another embodiment of any one of the above aspects, the hemp oil may comprise about 15% to 25% Omega 3, (ALA), about 50% to 60% Omega 6 (LA), about 1% to 5% Omega 6 (GLA), and about 10% to 15% Omega 9 (oleic acid), per 100 g of hemp oil.

In still another embodiment of any one of the above aspects, the hemp oil may comprise about 1 g to 5 g Omega 3, (ALA), about 5 g to 15 g Omega 6 (LA), about 0.2 g to 1 g Omega 6 (GLA), and about 1 g to 5 g Omega 9 (oleic acid), per 20 g of hemp oil.

In some embodiments of any one of the above aspects, the hemp oil may comprise about 3.5 g Omega 3, (ALA), about 11.2 g Omega 6 (LA), about 0.4 g Omega 6 (GLA), and about 2.5 g Omega 9 (oleic acid).

In other embodiments of any one of the above forms, the hemp oil may comprise about 3.3 g Omega 3, (ALA), about 10.7 g Omega 6 (LA), about 0.7 g Omega 6 (GLA), and about 2.7 g Omega 9 (oleic acid).

In other embodiments of any one of the above aspects, the hemp oil may have a ratio of Omega 3 to Omega 6 of between about 1:5.2 and 5:16. The hemp oil may have a ratio of Omega 3 to Omega 6 of about 3.5:11.6. The hemp oil may comprise a 1:3 ratio of omega 3 and 6.

In still other embodiments of any one of the above aspects, the hemp, hemp oil or a pharmaceutically effective extract is for use or when used as a carrier or delivery vehicle for the one or more steroids.

In still another embodiment, the hemp, hemp oil; or pharmaceutically effective extract comprises a form suitable for administration by one or more of oral, intradermal, intramuscular, intraperitoneal, parenteral, intravenous, subcutaneous, intranasal, epidural, sublingual, intracerebral, intravaginal, transdermal (e.g., via a patch), rectal, by inhalation, transmucosal, or topical, particularly to the ears, nose, eyes, or skin. The pharmaceutical composition may be injectable. The parenteral or injectable form may comprise any suitable form for parenteral or injectable administration such as an injectable solution, an injectable suspension, an injectable emulsion, and an injection in a form that is prepared at the time of use. Formulations for parenteral administration may be in a configuration such as an aqueous or nonaqueous isotonic aseptic solution or suspension. The injectable form may be for intravitreal injection.

In another particular embodiment of any above aspect, the hemp, hemp oil or a pharmaceutically effective extract is preservative free.

In another particular embodiment of any above aspect, the hemp, hemp oil or a pharmaceutically effective extract is prophylactic.

In a particular embodiment of any one of the above forms, the hemp, hemp oil or a pharmaceutically effective extract is sterilized.

Other aspects and preferred forms are disclosed in the specification and/or defined in the appended claims, forming a part of the description of the invention.

In essence, embodiments of the present invention stem from the realization that certain compounds are efficacious for conditions which are refractory to existing treatments.

Advantages provided by the present invention comprise the following:
Improved outcomes for treatment of eye conditions;
Increased options for treatment of eye conditions.

Further scope of applicability of embodiments of the present invention will become apparent from the detailed description given hereinafter. However, it should be understood that the detailed description and specific examples, while indicating preferred embodiments of the invention, are given by way of illustration only, since various changes and modifications within the spirit and scope of the disclosure herein will become apparent to those skilled in the art from this detailed description.

### BRIEF DESCRIPTION OF THE EXAMPLES

Further disclosure, objects, advantages and aspects of preferred and other embodiments of the present application may be better understood by those skilled in the relevant art by reference to the description and the following non-limiting Examples taken in conjunction with the accompanying drawings, which are given by way of illustration only, and thus are not limitative of the disclosure herein.

### Example 1

The efficacy and mechanism of FA and TA were studied with respect to modulation of Ccl2, Il-6, Il-8 expression levels in Muller cells, and their effect on retinal degeneration in the PD model. Full details of the study are included in the APPENDIX.

The study confirmed the novel anti-inflammatory and neuroprotective properties of TA and more particularly, FA, in the treatment of retinal degeneration.

Data shows that FA and TA and do not induce toxicity in multiple human retinal cell lines, including photoreceptor-like (661W), Muller cells (MIO-M1), and RPE (APRE19). Addition of either FA or TA drastically reduced the expression of Ccl2, Il-6, Il-8 in Muller cells stimulated with Il1b or TNFa, and which was dependent on glucocorticoid receptor signalling. Finally, administration of FA improves photoreceptor survival in PD, while TA had no significant effect.

### Example 2

To evaluate the safety and tolerability of a single-dose intravitreal (IVT) injection of 1 mg/0.1 mL and 2 mg/0.1 mL Fludrocortisone acetate (FCA) in subjects with geographic atrophy (GA) secondary to Age-Related Macular Degeneration (AMD).

Methods: This was a two-part dose-escalation prospective study. Part-1 involved a single participant treated with 1 mg/0.1 ml and monitored up to 28-days before being reviewed by a safety review committee. Two subsequent participants were then dosed with the same dose. Part-2 involved a single participant dosed with 2 mg/0.1 ml and monitored up to 28-days when a further 5 participants were dosed. All participants were followed up for 6-months after baseline.

A full ophthalmic assessment was performed at study visits which included GA area, best-corrected visual acuity (BCVA), low-luminance BCVA and intra-ocular pressure (IOP). Adverse events (AE) were reported from the first dose of FCA until the end-of-study visit.

Results: There were no serious AEs (ocular or systemic) observed with IVT FCA at either 1 mg/0.1 ml2 mg/0.1 ml among the 9 participants. There was no evidence of increased IOP or cataract development.

Neither BCVA or LL-BCVA changed significantly in the study-eye over the follow-up period (p=0.28 and 0.38 respectively). Mean GA area increased in the study (0.5 mm2 p=0.003) and fellow-eyes (0.62 mm2 p=0.02) over 6-months. Differences between eyes were not significant (p=0.64), and at the lower end of population norms.

Conclusion: Intravitreal FCA is clinically safe and well-tolerated and did not increase TOP and has promising indications in terms of GA lesion growth.

### BRIEF DESCRIPTION OF THE DRAWINGS

FIG. 1 illustrates the effect of FA and TA on the viability of retinal cells in vitro. The effect of FA (A-C) and FA (D-F) on cell viability was assessed using the MTT assay on immortalized human cells derived from Muller glia (MIO-M1), RPE (ARPE-19), and photoreceptors (661W).
A-C: The addition of FA had no significant effect on the viability of all cell lines assessed, compared to the DMSO-only group (P>0.05). The exceptions were MIO-M1 cells (A), which had a slight reduction at the highest concentration (10 ug/uL, P<0.05).
D-F: Introduction of TA similarly did not have significant effect on viability across all lines (P>0.05) except for MIO-M1 (D) and 661W cells (E), which had a slight reduction at 10 ug/uL (P<0.05).

Statistical significance was determined using a one-way ANOVA and Tukey's post-hoc test. Results presented as the mean±SEM.

FIG. 2 illustrates modulation of pro-inflammatory cytokine expression by FA and TA in vitro. The effect of FA and TA on induction of Ccl2, Il-6, and Il-8 expression by MIO-M1 cells was assessed following their stimulation with either IL-1β (A-B) or TNF (C-D).
A-B: After stimulation with IL-1β, MIO-M1 cells exhibited profound increases Ccl2, Il-6, and Il-8 expression (P<0.05). When FA was added to the culture media upon IL-1β stimulation however (A), the up-regulation of Ccl2, Il-6, and Il-8 was suppressed to near baseline levels (P<0.05). A significant reduction in the expression of Ccl2, Il-6, and Il-8 was also observed in groups where TA was instead added (P<0.05).
C-D: Stimulation with TNF induced a similar, though less pronounced, up-regulation of Ccl2, Il-6, and Il-8 in MIO-M1 cells (P<0.05). This effect was also significantly inhibited when FA or TA were added to the culture media (P<0.05).

Statistical significance was determined using a one-way ANOVA and Tukey's post-hoc test. Results presented as the mean±SEM.

FIG. 3 Contribution of glucocorticoid and mineralocorticoid receptor signalling to the anti-inflammatory effect of FA in vitro. The inhibitory effect of FA on Ccl2, Il-6, Il-8 expression in Il-1b-stimulated MIO-M1 cells was further investigated through co-incubation with selective antagonists (A-D).
A-B: The addition of RU486, a glucocorticoid receptor antagonist (A), was found to completely abrogate the FA-mediated suppression of Ccl2, Il-6, and Il-8 following IL-b simulation (P<0.05). However, incubation with RU28318, a mineralocorticoid receptor antagonist (B), did not significantly alter the expression of Ccl2, Il-6, and Il-8, compared to MIO-M1 cells treated with Il1b and FA alone (P>0.05).
C-D: Incubation of antagonists to either the androgen receptor, PF998425 (C), or the estrogen receptor, ICI182780 (D), did not alter the suppression of Ccl2, Il-6, and Il-8 by FA following Il1-b stimulation (P>0.05).

Statistical significance was determined using a two-way ANOVA and Tukey's post-hoc test. *denotes a significant change in comparison to FA+IL-1β. Results presented as the mean±SEM.

FIG. 4 illustrates the neuroprotective properties of FA and TA on retinal cell death in mice following PD. Effect of intravitreal injection of either FA or TA on photoreceptor degeneration following PD was determined with a combination of OCT segmentation (A), ERG recordings (B), and abundance of apoptotic TUNEL+ photoreceptors (C).
A-C: ONL measurements quantified from OCT images centred on the lesion area (1-2/2-3 mm eccentricity from the optic nerve) revealed a significant preservation in ONL thickness in the FA treated group (B) compared to controls following PD (C, P<0.05). In contrast, there was no significant change in the TA-treated group (P>0.05).
D-E: FA-treated mice showed significantly larger a-wave (D) and b-wave (E) ERG responses compared to both control and TA-treated groups (P<0.05). F-H: Following exposure to PD, mice treated with FA were found to have significantly fewer TUNEL+ photoreceptors than vehicle-treated mice (P<0.05). Conversely, mice treated with TA exhibited no significant change in the number of TUNEL+ photoreceptors (P>0.05). Statistical significance was determined using either one-way ANOVA (A, F, Tukey's post-hoc test) or two-way ANOVA (D, E, Tukey's post-hoc test).

Scale bar represents 50 μm. GCL (ganglion cell layer); INL (inner nuclear layer); ONL (outer nuclear layer).

FIG. 5 illustrates the effect of FA and TA on macrophage infiltration in mice following PD in mice. The infiltration of activated macrophages to the outer retina following PD was quantified (A) using immunolabeling for IBA1 (B-C, green).
A-C: There was an abundance of IBA1+ macrophages within the outer retina following PD (A), which coincided spatially with disruption to the ONL and subretinal space (B, arrows). In contrast, there was significant reduction of the number IBA1+ cells in both FA- and TA-treated groups compared to control, with FA showing the most pronounced trend (P<0.0001).

Statistical significance was determined using one-way ANOVA. Scale bar represents 50 μm. GCL (ganglion cell layer); INL (inner nuclear layer); ONL (outer nuclear layer).

FIG. 6: Example of atrophy as measured by Heidelberg Region Finder software (A) baseline multicolor, (B) baseline fluorescein angiogram, (C) baseline autofluorescence, (D) baseline region finder, (E) month 1, (F) month 2, (G) month 3 and (H) month 6.

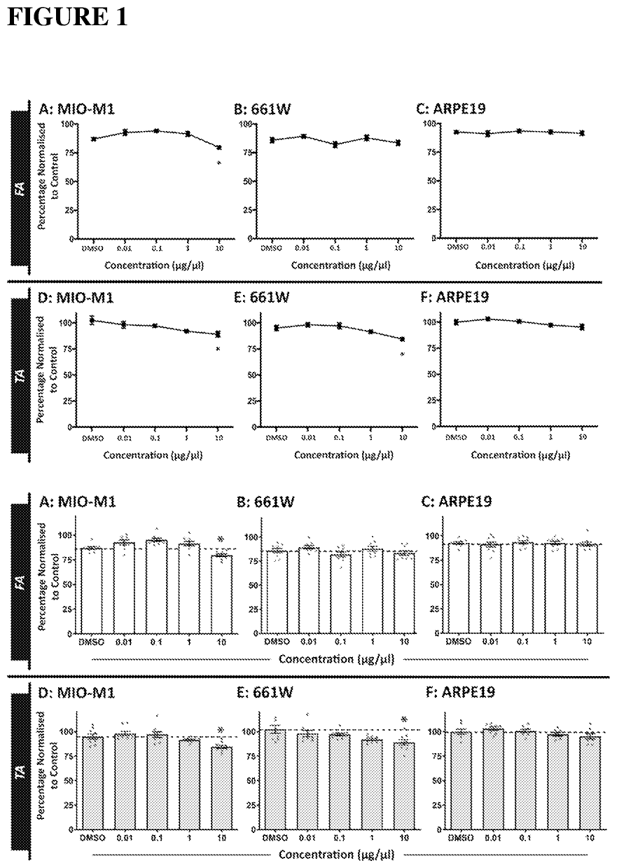

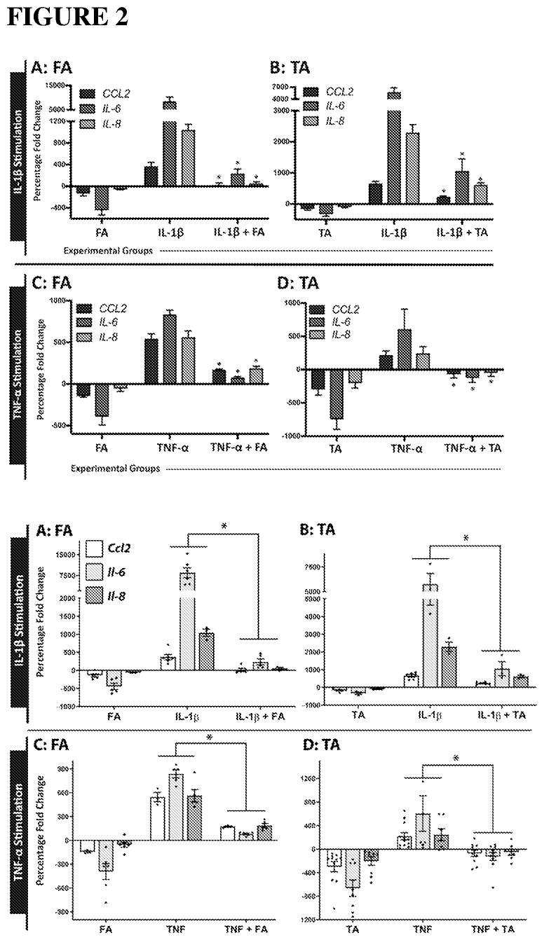

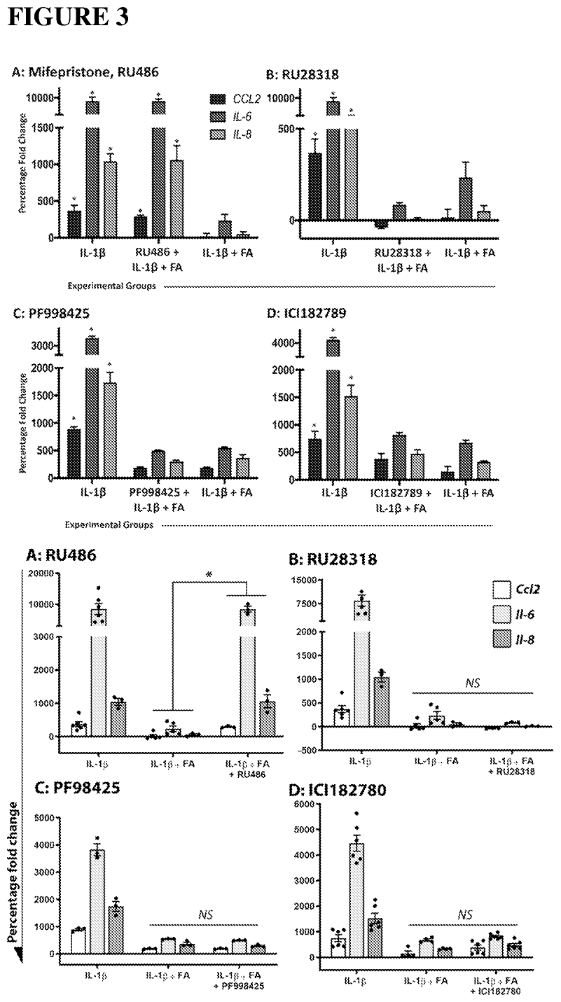

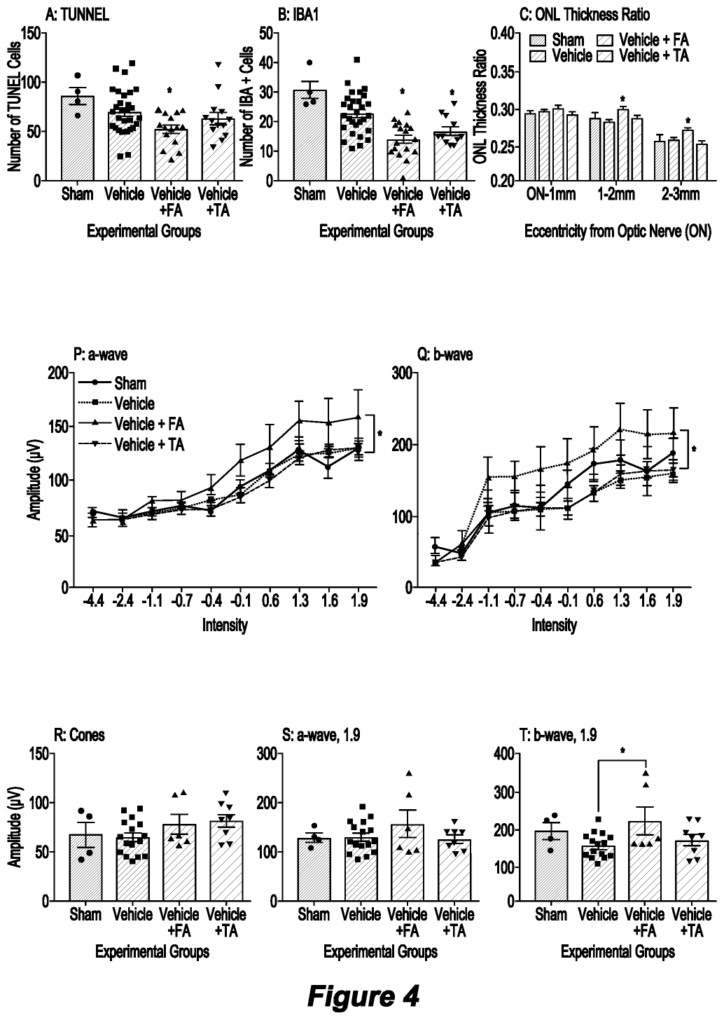

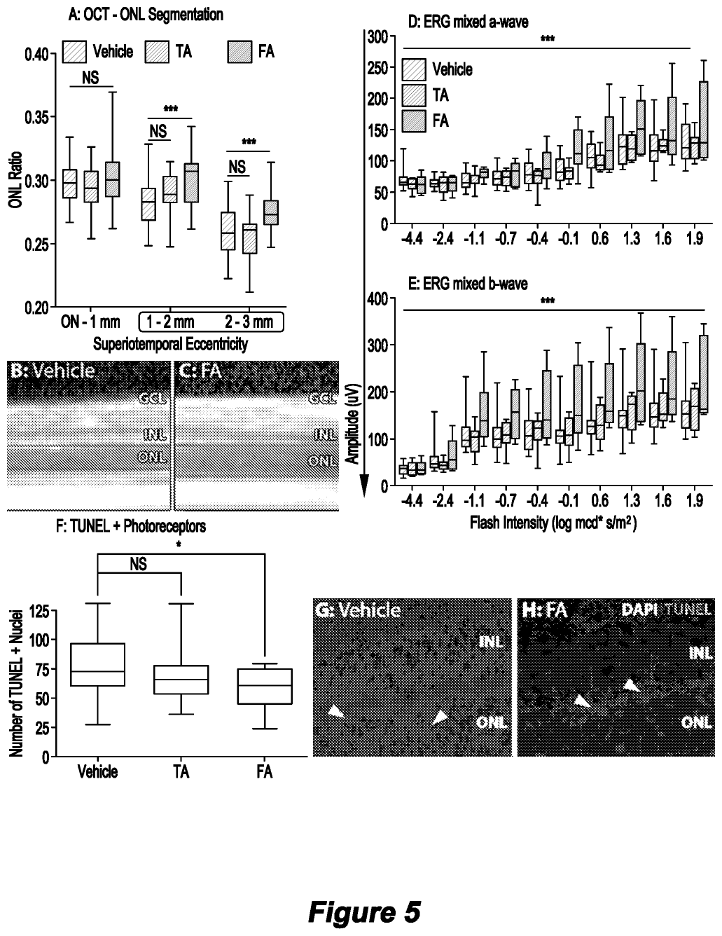

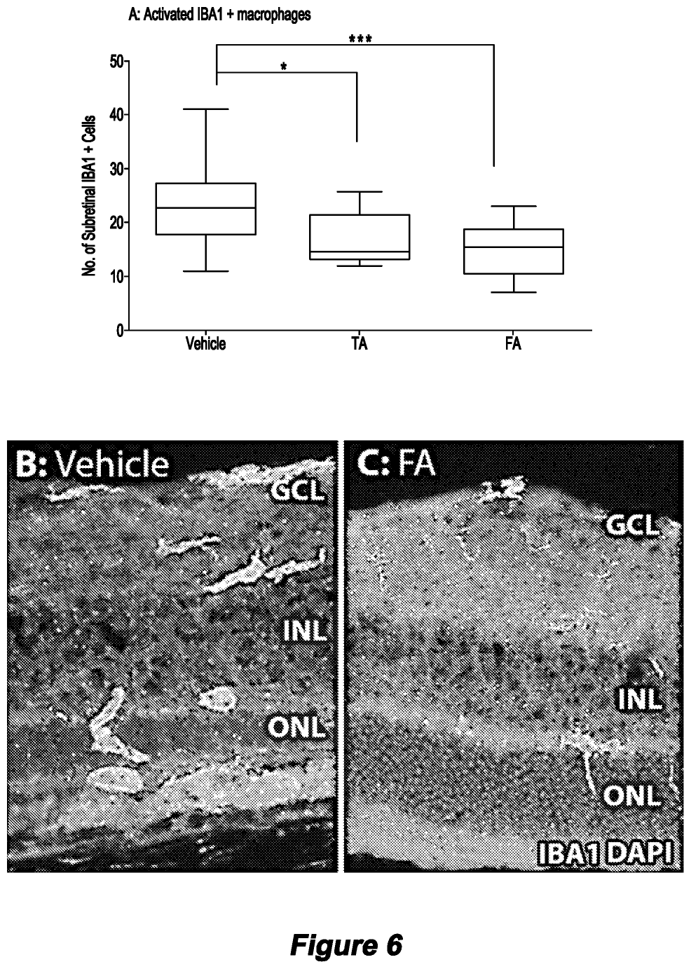

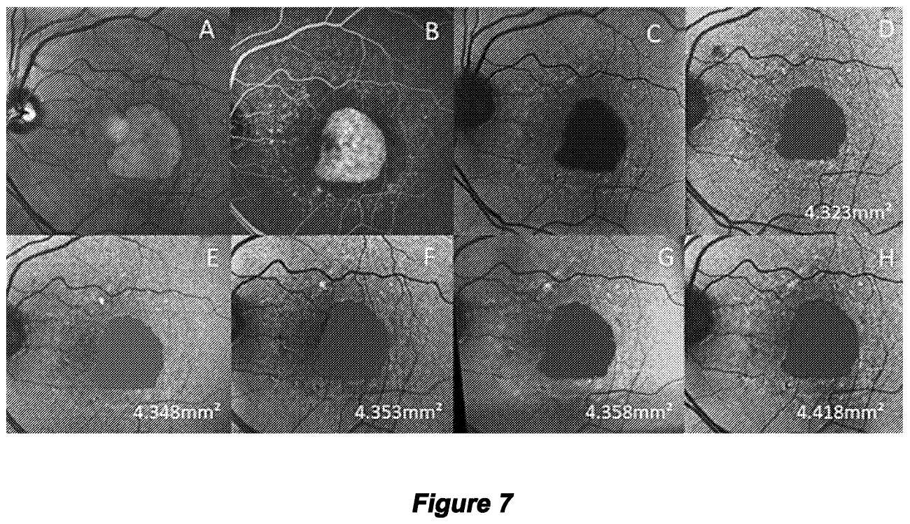

### DETAILED DESCRIPTION

The present invention thus provides a method for treating a subject having one or more retinal eye condition that is refractory to anti-VEGF treatments comprising administering to the subject a therapeutically effective amount of one or more steroids, preferably mineralocorticoids or glucocorticoids, subsequent to anti-VEGF treatment, thereby treating the one or more retinal eye condition.

Steroids suitable for administration include, but are not limited to: cortisol, cortisone, prednisone, prednisolone, methylprednisolone, dexamethasone, triamcinolone (Aristocort™, Kenalog™), betamethasone (Celestone™), budesonide, fluormetholone (fluorometholone acetate (Flarex™, Eflone™), fluormetholone alcohol (FML™ FML-Mild™, FluorOP™), medrysone alcohol (HMSO), loteprednol etabonate (Lotemax™ Alrex™) and anecortave acetate (Alcon™), beclometasone, fludrocortisone, deoxycorticosterone, aldosterone, triamcinolone acetonide (TA), 11-desoxycortisone (11-DC), fludrocortisone (FA), deoxycorticosterone acetate (DA), deoxycorticosterone (DS), fluocinolone or a therapeutically active analogue, derivative, homolog, pharmaceutically acceptable salt or conjugate thereof. It will be appreciated that the above list is representative only and is not exclusive.

The therapeutically effective amount of one or more compounds may be capable of modulating an activity of a steroid receptor, preferably a glucocorticoid receptor and/or mineralocorticoid receptor, subsequent to anti-VEGF treatment, thereby treating the retinal eye condition.

The one or more compounds may comprise one or more dual action compounds, wherein each dual action compound is capable of modulating the activity of both a mineralocorticoid receptor and a glucocorticoid receptor. The dual action compound may for example, comprise one or more of cortisol; cortisone; prednisone; prednisolone; methylprednisolone; fludrocortisone acetate; deoxycorticosterone acetate; aldosterone or a therapeutically active analogue, derivative, homolog, pharmaceutically acceptable salt or conjugate thereof.

### Mineralocorticoids

In a preferred embodiment, the one or more mineralocorticoids may comprise one or more of: triamcinolone acetonide (TA); 11-desoxycortisone (11-DC); fludrocortisone (FA); Deoxycorticosterone acetate (DA); Deoxycorticosterone (DS); or Aldosterone; or a therapeutically active analogue, derivative, homolog, pharmaceutically acceptable salt or conjugate thereof. A homolog comprises a molecule of the same chemical type but differing by a fixed increment of an atom or a constant. The mineralocorticoid may further comprise one or more pharmaceutically acceptable carriers, diluents or excipients.

In a particularly preferred embodiment, the mineralocortico steroid is of the general structure:

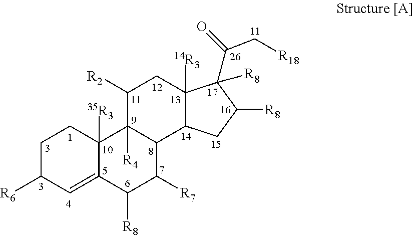

wherein
R₁ is CH₃, CH═O, CH₂OH, CR═O, CH₂NH₂, COCHCH₂, NO₂, X or CH₂X wherein X is F, Cl, Br;
R₂ is H, OH, ═O, NH₂ or CN;
R₃ is H, CH₃ or CH₂OH;
R₄ is H, F or Cl;
R₅ is H, OH, ═O, SH, NH₂CN, NO₂, CH₃, CH₂OH, CH═O, X, CH₂X, OR, OCOR, OPO(OR)₂, ═CH₂ or CHR wherein X is F, Cl, Br;
R₆ is H, CH₃, or X wherein X is F, Cl, Br;
R₇ is H, OH, CH₃, Alkyl, Ph, X, OCH₃, OR, SCOCH₃, SCOR, OCOR, CH₂OH, CH₂X, CH═O, CR═O or NHCOR wherein X is F, Cl, BR;
R₈ is H, OH, CH₃, CH₂CH₃, SH, NH₂ or X wherein X is F, Cl, Br;
R₉ is H, OH, SH, NH₂, CH₃, CH₂CH₃ or X wherein X is F, Cl, Br;
R₁₀ is H, OH, CH₃, CH₂CH₃, SH, NH₂, CH₂OH, CH₂CH₂OH, OR, OCOR, OPO(OR)₂, NHCOR, CH═O, X or CH₂X wherein X is F, Cl, Br.

In a particularly preferred embodiment, the mineralocortico steroid is of the general structure:

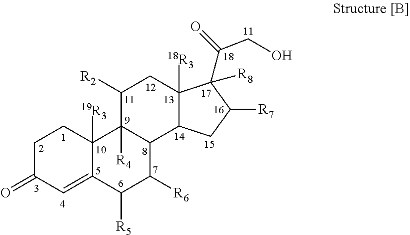

wherein
R₁ is CH₃, CH═O, or COCHCH₂;
R₂ is H, OH or ═O;
R₃ is H, CH₃ or CH₂OH;
R₄ is H or F;
R₅, R₆ and R₇ are H; and
R₈ is H or OH.

In a particularly preferred embodiment, the mineralocortico steroid is of the general structure:

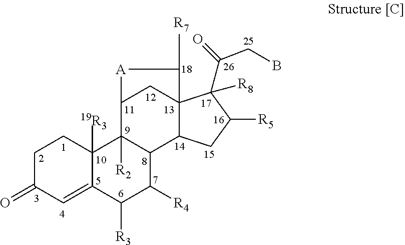

wherein
A is O, S, NH, CH₂, CHOH, C═O, CHX, CHCH₃, CH₂CH₂, OCH₂, SCH₂, NHCH₂, NRCH₂, NCOR wherein X is F, Cl, Br;
B is H, OH, SH, NH₂, CH₂OH, CH₂CH₂OH, CH═O, X, CH₂X, OR, OCOR, OPO(OR)₂, NHCOR wherein X is F, Cl, Br;
R₁ is H, CH₃, CH═O, CH₂OH;
R₂ is H, F, or Cl;
R₃ is H, CH₃, or X wherein X is F, Cl, Br;
R₄ is H, OH, CH₃, Alkyl, Ph, X, OCH₃, SH, NH₂, OR, SCOCH₃, SCOR, OCOR, CH₂OH, CH₂X, CH═O, CR═O, SR or NHCOR wherein X is F, Cl, Br;
R₅ is H, CH₃, CH₂CH₃, OH, SH, NH₂ or X wherein X is F, Cl, Br;
R₇ is H, OH, SH, NH₂, NO₂, CH═O, CH₃, CO₂H, CN, CH₂CH₃, CH₂X or X wherein X is F, Cl, Br.

### Glucocorticoids

In a preferred embodiment the glucocorticoids may comprise one or more of: cortisol, cortisone, prednisone, prednisolone, methylprednisolone, dexamethasone, betamethasone, triamcinolone (Aristocort™, Kenalog™), beclomethasone (Celestone™) fludrocortisone, deoxycorticosterone, aldosterone, fluocinolone or a therapeutically active analogue, derivative, homolog, pharmaceutically acceptable salt or conjugate thereof.

A homolog comprises a molecule of the same chemical type but differing by a fixed increment of an atom or a constant. The glucocorticoid may further comprise one or more pharmaceutically acceptable carriers, diluents or excipients.

### Formulation

An effective quantity of the compound of interest is preferably employed in the method of the invention. For formulations, the concentration of the therapeutic compound may be in the range of about 0.01 wt % to about 10 wt %. Typically, the concentration is in the range of about 0.025 wt % to about 2.5 wt %.

The precise pharmaceutical formulation used in the method of the present invention will vary according to a wide range of commercial and scientific criteria. The skilled reader will appreciate that the invention described above may contain other agents.

For example, the formulations used are preferably prepared using a physiological saline solution as a vehicle. The pH of the formulation may be maintained at a substantially neutral pH (for example, about 7.4, in the range of about 6.5 to about 7.4) with an appropriate buffer as known to one skilled in the art. Any pharmacologically acceptable buffer suitable for application to the eye may be used, such as, tris or phosphate buffers, acetate buffers, citrate buffers, phosphate buffers or borate buffers. Suitable water soluble buffering agents that may be employed are sodium carbonate, sodium borate, sodium phosphate, sodium acetate, sodium bicarbonate.

These agents may be present in amounts sufficient to maintain a pH of the system. As such, the buffering agent may be as much as about 5 wt % of the total formulation.

Any diluent used in the preparation of the pharmaceutically acceptable formulation may preferably be selected so as to not unduly affect the biological activity of the formulation. Examples of diluents which are especially useful for injectable formulations are water, organic or inorganic salt solutions, Ringer's solution, dextrose solution and Hank's solution.

In addition, the pharmaceutical formulation used in the method of the invention may include additives such as other buffers, diluents, carriers, adjuvants or excipients.

Other agents may be employed in the formulation for a variety of purposes. These include, for example, preservatives, co-solvents, surfactants, oils, humectants, emollients, chelating agents, stabilizers, tonicity adjustors or antioxidants. Water soluble preservatives which may be employed include, but are not limited to, benzalkonium chloride, chlorobutanol, thimerosal, sodium bisulphate, phenylmercuric acetate, phenylmercuric nitrate, ethyl alcohol, methylparaben, polyvinyl alcohol, benzyl alcohol and phenylethyl alcohol. A suitable surfactant may be, for example, Tween 80.

Other agents that may be used include, but are not limited to, polyvinyl alcohol, povidone, hydroxypropyl methyl cellulose, polosamers, carboxymethyl cellulose, hydroxyethyl cellulose, purified water. Tonicity adjustors may be included, such as, sodium chloride, potassium chloride, mannitol and glycerin. Antioxidants include, but are not limited to, sodium metabisulphite, sodium thiosulphate, acetylcysteine, butylated hydroxyanisole, butylated hydroxytoluene. The indications, effective doses, formulations, contraindications, vendors etc of the compounds in the formulations are available or are known to one skilled in the art.

These agents may be present in individual amounts of from about 0.001 wt % to about 5 wt % and preferably about 0.01 wt % to about 2.0 wt %.

### Administration

In performing the method of the invention, pharmaceutically acceptable compounds may be administered to a patient by any method that leads to delivery of the therapeutic compound to the site of the condition affecting the retina of the eye. Any of the formulations may be administered by an ocular route, such as topical (extraocular application), subconjunctival, sub-Tenon, intraocular, via ocular implants, or systemically (oral or another parenteral route). Administration of the composition is preferably by intraocular injection although other modes of administration may be effective.

In a highly preferred form of the invention, when administering the compound by intravitreal injection for treatment of a condition of the retina of an eye, the active compound(s) should be concentrated, as feasible, to minimise the volume for injection.

Preferably the compositions are administered in unit dosage forms suitable for single administration of precise dosage amounts. For example, the compositions of the present invention may be provided in the form of a single unit dose in a pre-prepared syringe, ready for administration.

Solid dispersions of the therapeutic compound as well as solubilised preparations can be used to perform the treatment method of the invention. For intraocular formulations, the therapeutic compound is delivered at a concentration high enough to achieve a final concentration in the range of about 0.1 μmol/L to about 10 μmol/L within the target ocular compartment (such as the posterior chamber for the treatment of retinal diseases). Typically for this mode of delivery the final concentration of the therapeutic compound is in the range of 0.25 μmol/L to about 5 μmol/L.

Topical application of formulations of the invention may be as an ointment, gel or eye drops or a formulation that gels in situ, upon contact with the eye or with lacrimal fluid in the exterior of the eye. For topical administration, the concentration of the therapeutic compound administered may depend on the particular subject, the underlying disease and its severity, the dosing frequency and other factors as known to one skilled in the art. Sample concentrations include, but are not limited to, about 0.5 μg/ml to about 500 μg/ml, more preferably about 1.0 μg/ml to about 100 μg/ml, even more preferably about 5 μg/ml to about 50 μg/ml.

The formulation may also be administered as a slow release formulation, with a carrier such as microspheres, microcapsules or liposomes as a topical ointment or solution, an intravenous solution or suspension, or in an intraocular injection, as known to one skilled in the art.

A time-release drug delivery system may be administered intraocularly to provide sustained release of the formulation over a period of time. For example, a slow or extended release delivery system may be provided in the form of an implant.

FA has been shown to be efficacious in the treatment of DME and other wet retinal diseases. FA has also been tested in a safety study in Geographic Atrophy. MA arises as a result of long-term treatment with anti-VEGFs. It is intended to test FA primarily, and possibly also TA, in DME that are refractory to Eylea and other anti-VEGFs.

### Appendix (Methods and Results for Example 1)

### Methods

### In Vitro Experiments

The immortalised retinal cells used were as follows: MIO-M1 cells (Muller cell-derived; Müller 1 Moorefields, Institute of Ophthalmology, Dr A. Limb, Institute of Ophthalmology, University College, UK); ARPE19 (RPE-derived; ATCC CRL-2302, American Tissue Culture Collection, VA, USA); and 661W cells (photoreceptor-like derived; kindly gifted by Dr Muayyad R. Al-Ubaidi, Department of Cell Biology, University of Oklahoma Health Sciences Centre, Oklahoma City, OK, USA). Cells were authenticated and validated from Cell Bank Australia.

### MTT Assay

The immortalised retinal cell lines MIO-M1, 661W and ARPE19 were used to undertake the MTT assay to assess cell metabolic activity, based on the cell's availability of NAPDH. Treatments included exposure to one of FA (10-0.01 μg/μl, provided by Professor Ben Boyd, Monash University, Australia), TA (10-0.01 μg/p, Honeywell, USA) and Dexamethasone (10-0.0001 μg/μl, Sigma-Aldrich, MO, USA). All corticosteroids used in vitro were dissolved in 20% dimethyl sulfoxide (DMSO) (Sigma-Aldrich, MO, USA) and ultrapure endotoxin 0.1M PBS (Thermo Fisher Scientific, MA, USA). At the 12-hour time point following addition of the corticosteroid, 10p of MTT reagent (Roche, Switzerland) was added to each well. To protect against light, plates were wrapped in foil and stored in the dark at 37° C. for 4 hours at 5.0% CO₂. Cells were monitored using a bright field Zeiss Axiovert 200 inverted microscope (Carl Zeiss Meditec, Dublin, CA. USA) for formation of insoluble purple formazan precipitate crystals in viable cells. Subsequently 100p of MTT solubilisation solution was added and once again incubated in darkness at 37° C., 5.0% CO₂ for 24 hours. Quantification of the MTT assay was performed by measuring the absorbance at 570 nm using Tecan Infinite® 200 PRO (Tecan, Mannedorf, Switzerland).

### IL-1β and TNF-α Stimulation

IL-1β and TNF-α were administered to MIO-M1 cells to initiate an inflammatory response in order to test the anti-inflammatory properties of the selected corticosteroids. MIO-M1 cells were grown to a cell density of 50,000 cells per well in a 24 well plate using growth media and foetal bovine serum as described (Section 2.1.1). MIO-M1 cells were challenged with either 1 ng/μl of IL-1β or TNF-α (R&D Systems, MN, USA). Experimental wells were treated with either 1 μg/μl FA, TA or Dexamethasone. Twelve hours post addition, growth medium was removed from each well and replaced with 860 μl of TRIzol (Thermo Fisher Scientific, MA, USA), agitated approximately 20 times and then placed in a clean 1.5 ml Eppendorf tube to extract ribonucleic acid (RNA).

### Animal Handling and Rearing

All experiments conducted were in accordance with the ARVO Statement for Use of Animals in Ophthalmic and Vision Research, and with approval from the ANU Animal Experimentation Ethics Committee (AEEC, protocol #A2014/56). C57BL/6J mice aged 60 post-natal days were used in this study. Animals were born, reared and held under dim light conditions (5 lux) prior to photo-oxidative damage.

### Optical Coherence Tomography (OCT)

In vivo cross-sectional and fundus images of the mouse retina were acquired using Optical Coherence Tomography (OCT).

### Intravitreal Injections

Intravitreal injections were performed on C57BL/6J mice aged 60 post-natal days. Treatments included exposure to FA or TA dissolved in Suspension (comprised of 0.5% w/v carboxymethyl cellulose and 0.4% v/v Tween 80). The final concentration of drug in the vitreous chamber is estimated to be as 1 mg/ml.

### Photo-Oxidative Damage in the Mouse Paradigm

Age-matched C57BL/6J mice (60 post-natal days) were exposed to continuous 100K lux white LED light for 5 days with access to food and water ad libitum. During this time, 1% atropine sulphate (Minims 1% atropine sulphate eye drops; Bausch and Lomb, USA) was administered to both eyes twice daily to dilate the pupils. Animals were subsequently euthanised with carbon dioxide (CO₂).

### Electroretinography (ERG)

Electroretinography (ERG) was used to assess the retinal function response of rodents to full-field flash stimuli under scotopic conditions. Dim-reared control and photo-oxidative damaged animals were used, using known methods.

Animals were dark-adapted overnight before the commencement of ERG experiments. Animals were anaesthetised as previously described, and all retinal recordings were undertaken in a dark room with minimal red light source.

### TUNEL

Terminal deoxynucleotidyl transferase (Tdt) dUTP nick end labelling (TUNEL) is an established technique used to detect apoptotic and necrotic cells.

### Immunohistochemistry

Details of the primary and secondary antibodies are listed in Table 1.

TABLE 1
Primary and Secondary Antibodies
used for immunohistochemistry.

| Antibody | | | Optimal | |
|---|---|---|---|---|
| Type | Name of Antibody | Species | Dilution | Supplier |
| Primary | IBA1 (ionised | Rabbit | 1:500 | Wako, Japan |
| | calcium binding | | | |
| | adaptor molecule 1) | | | |
| Secondary | Alexa Fluor Goat | Goat | 1:500 | Thermo Fisher |
| | Alpha Rabbit 488 | | | Scientific, MA, |
| | | | | USA |

### Quantification of TUNEL and IBA1 Positive Cells

The retina was evenly divided into 8 quadrants. Each quadrant was viewed using a confocal microscope (LSM 5, Zeiss, Germany) and TUNEL⁺ and IBA1⁺ cells were identified and counted. Data were graphed and analysed using Prism 6 software (GraphPad Software, CA, USA).

### Quantitative Real Time Polymerase Chain Reaction

Following purification of RNA, cDNA was synthesized using the Bioline Tetro kit (Bioline, Australia) as per the manufacturer's instructions. Quantitative Real Time Polymerase Chain Reaction (QRT-PCR) was undertaken to profile gene expression changes in animal and cell culture samples. TaqMan hydrolysis probes (Applied Biosystems, USA) and Gene Expression Master Mix (Applied Biosystems, USA) were used. A clear 384 well plate (Applied Biosystems, USA) was used with each biological sample plated in duplicate. A reaction mixture per sample was set up and included 0.5 μl cDNA, 4 μl Rnase Free Water, 5 μl Gene Expression Master Mix and 0.5 μl Taqman Primer Probe (Thermo Fisher Scientific), as listed in Table 2.5. The amplification of each sample was undertaken using a QuantStudio 12K Flex QRT-PCR machine (Applied Biosystems, USA). GAPDH was used as the reference gene using the comparative cycle threshold (Cₜ) method (ΔΔCₜ), as it is known not to change expression following retinal photo-oxidative damage. Data was analysed using the QuantStudio 12K Flex Software (Applied Biosystems, USA).

TABLE 2
TaqMan Hydrolysis Probes used for qPCR.

| | | | Catalogue |
|---|---|---|---|
| Gene | Name | Type | Number |
| C3 | Complement component 3 | Mouse | Mm00437858_m1 |
| CCL2 | Chemokine (C-C motif) ligand 2 | Human | Hs00234140_m1 |
| | | Mouse | Mm99999056_m1 |
| CCL12 | Chemokine (C-C motif) ligand 12 | Mouse | Mm01617100_m1 |
| GAPDH | Glyceraldehyde-3-phosphate | Human | Hs02758991_g1 |
| | dehydrogenase | Mouse | Mm99999915_g1 |
| | (Housekeeping Gene) | | |
| IL-1β | Interleukin 1ß | Mouse | Mm00434228_m1 |
| IL-6 | Interleukin 6 | Human | Hs00174131_m1 |
| | | Mouse | Mm00446190_m1 |
| IL-8 | Interleukin 8 | Human | Hs99999034_m1 |
| SDHA | Succinate Dehydrogenase Complex | Mouse | Mm0152366_m1 |
| | Flavoprotein Subunit A | | |
| | (Housekeeping Gene) | | |
| TNF | Tumour necrosis factor | Mouse | Mm00443258_m1 |

### Statistical Analysis, Image Acquisition and Presentation

All data was analysed using Prism 6 software (GraphPad Software, CA, USA). Analyses included a one-way analysis of variance (ANOVA) and unpaired student t-tests, with appropriate post hoc tests as indicated. Results were presented as standard error of the mean. Statistical significance was considered P<0.05. All images were taken using an A1 Nikon Confocal Microscope (Nikon, Toyko, Japan). Images were acquired using the NIS-Elements AR Software (Nikon, Tokyo, Japan) and uniformly processed using Photoshop CS6 (Adobe Systems, CA, USA).

### Results

### Cytotoxicity of Corticosteroids on Retinal Cells

The effects of FA and TA on the viability of MIO-M1, 661W and ARPE19 cells indicate that that 1 μg/μl does not significantly alter cell viability across these lines via the MTT assay (FIG. 1, P<0.05). Thus, all subsequent experiments were undertaken using this dose of FA and TA, respectively.

### FA and TA Modulate Chemokine and Cytokine Expression

When challenged with the pro-inflammatory stimulus IL-1β the gene expression of three key inflammatory markers CCL2, IL-6 and IL-8 is significantly increased in MIO-M1 cells (FIG. 2, P<0.05). FIG. 2 shows the responses of MIO-M1 cells challenged with IL-1β, which results in the upregulated expression of CCL2, IL-6 and IL-8 genes. Treatment with FA returned the gene expression levels of CCL2, IL-6 and IL-8 to baseline levels (FIG. 2A, P<0.05). Similar levels of CCL2, IL-6 and IL-8 upregulation were observed in MIO-M1 cells following stimulation with TNF-α and were significantly reduced by treatment with FA (FIG. 2C, P<0.05).

Treatment of the stimulated cells with TA significantly reduces the expression of CCL2, IL-6 and IL-8 to around one-third of the stimulated levels (IL-1β alone) (FIG. 2B, P<0.05). A similar pattern of response in CCL2, IL-6 and IL-8 gene expression was observed when TNF-α was used as the pro-inflammatory stimulus, with gene expression levels of the selected inflammatory markers also falling significantly after addition of TA (FIG. 2D, P<0.05).

### FA Modulates Chemokine and Cytokine Expression Through the Mifepristone/(RU486)-Sensitive Glucocorticoid Receptor

Inclusion of receptor-blockers found that the impact of FA on expression of the target genes was negated in the presence of the glucocorticoid receptor blocker, Mifepristone (RU486), but not in the presence of RU28318 (MR blocker), PF998425 (androgen blocker) or ICI182780 (oestrogen blocker) (FIG. 3). The results indicate that the impact of FA on expression of CCL2, IL-6 and IL-8 is mediated through the glucocorticoid receptor. This pattern was also observed for TA.

### FA is a Protective and Anti-Inflammatory Agent in a PD Model of Retinal Degeneration

Compared to controls, Suspension+FA injected mice had significantly fewer TUNEL⁺ cells in the outer retina at 5 days PD, unlike mice injected with Suspension alone, and Suspension+TA (P<0.05, FIG. 4A). There was a significant decrease in IBA1⁺ cells in both Suspension+TA and Suspension+FA injected mice compared to controls (P<0.05), but not in mice injected with Suspension vehicle alone (FIG. 4B). Suspension+FA injected mice had a significantly greater ONL thickness ratio in the locations 1-2 mm and 2-3 mm superior to the optic nerve, compared to controls (P<0.05, FIG. 4C), a difference which was detectable by OCT (FIG. 4D-G). This effect was not observed in Suspension+TA injected animals. Histological findings are shown in FIGS. 4H-O. ERG analysis shows that mice injected intravitreal with Suspension+FA had significantly higher a- and b-wave responses following 5 days PD (P>0.05). Mice injected with the Suspension-alone or Suspension+TA had ERG responses that were indistinguishable from PD controls/untreated animals (FIG. 4P-Q). There were no significant differences in the cone response between experimental groups (FIG. 5.4R).

Collectively, the key measures investigated including photoreceptor cell death, microglia/macrophage recruitment, ONL thickness and retinal function have contributed to assessing the efficacy of corticosteroids following PD.

### Appendix (Methods and Results for Example 2)

Age-related macular degeneration (AMD) is the leading cause of severe vision loss in people over the age of 65 in Western countries (1). In the United States, about 1.75 million people have the advanced forms of AMD (2, 3). The early signs of AMD (drusen and pigmentary changes) are common in individuals over age 65 and precede the advanced forms, which are visually devastating. The advanced forms of AMD are classified into either choroidal neovascularization (wet, or exudative) or geographic atrophy (dry).

Geographic Atrophy (GA) is a disease characterized by thinning and loss of the retinal pigment epithelium (RPE), and concurrent atrophy of photoreceptors and choriocapillaris (4). Clinically, GA is characterized by islands of dead retinal cells in the back of the eye that gradually expand. Although GA can result in significant visual function deficits in reading, night vision, and dark adaptation, and produce dense, irreversible scotomas in the visual field, the initial decline in VA may be relatively limited if the fovea is spared. When the fovea is involved, GA quickly causes blindness. GA is responsible for approximately 20% of all legal cases of blindness in North America with increasing incidence and prevalence owing to a higher life expectancy (5).

AMD is a highly complex disease that is affected by multiple factors, such as ageing, genetic predisposition, environmental elements, oxidative stress and inflammatory effects (2, 6, 7). Smoking, age, alcohol consumption, diet and obesity are important risk factors related to oxidative stress (7, 8). High body mass index, cardiovascular disease, hypertension and a variety of dietary patterns are risk factors less consistently (9). Several single-nucleotide polymorphisms (SNPs) that confer increased or decreased risk of inflammation have been identified. They include the well-recognized complement factor H (CFH), CX3CR1, Toll-like receptor 3 (TLR3), TLR4, and interleukin 8 (IL-8)(10).

Although AMD is not a classic inflammatory disease, inflammatory cells have an important role in AMD pathogenesis and progression (6, 11, 12). Evidence has also suggested that some infectious agents are associated with AMD. Interleukin 6 (IL-6) has also been found to be upregulated in neovascular AMD and GA, furthermore it has been linked to GA progression (13).

Fludrocortisone acetate (9-α-Fluoro-11 β. 17 α, 21-trihydroxy-4-pregnene-3, 20 dione acetate, FCA) is a synthetic steroid possessing a potent mineralocorticoid effect and high glucocorticoid activity (14), and so has anti-inflammatory and anti-allergic properties. FCA is a mineralocorticoid receptor and glucocorticoid receptor agonist that binds to cytoplasmic receptors, translocates to the nucleus and subsequently initiates the transcription of glucocorticoid-responsive genes such as lipocortins to inhibit phospholipase A2. This prevents the release of arachidonic acid, a precursor to prostaglandins and leukotrienes, both important mediators in the pro-inflammatory response mechanism. In addition, this agent exerts its mineralocorticoid effect on the distal tubules and collecting ducts of the kidney by inducing permease, an enzyme that regulates Na+ permeability in cells, thereby enhancing Na+ reabsorption and water retention as well as increasing K+, H+ excretion.

The purpose of this study was to assess the safety and tolerability of a single intravitreal (IVT) dose of FCA among patients with GA over a 6-month period.

### Methods

### Study Design

This single-centre, phase 1b prospective, open-labeled, single-dose, dose-escalation clinical trial was conducted on 9 participants enrolled at a single site between August 2019 to April 2021. All patients were followed up for 6 months after baseline. Ethics approval was obtained before commencement and the trial was listed on the Australian and New Zealand Clinical Trial Registry (accessible via www.anzctr.org.au; ANZCTR no. 12618001308280). An independent data and safety committee provided oversight of the clinical trial. This study adhered to the tenets of the Declaration of Helsinki. Informed consent was obtained from all participants before enrolment into the study.

### Study Medication

Fludrocortisone acetate was formulated for intravitreal administration as a powder solution for injection to ensure long-term stability similar to other cortico-steroids (15). Vials contained 10 mg of FCA powder and were reconstituted with sterile sodium chloride solution (0.9%) according to the appropriate dosage prior to injection.

### Study Population

Inclusion criteria referring the study eye were as follows: diagnosis of GA secondary to AMD confirmed using fundus autofluorescence (FAF) imaging, GA area between ≥1.9 and ≤17 mm2 (1 and 7 disc areas (DA) respectively), best corrected visual acuity (BCVA) of 24 letters or better using Early Treatment Diabetic Retinopathy Study (ETDRS) charts.

Exclusion criteria were as follows: GA due to causes other than AMD such as Stargardt disease, cone rod dystrophy or toxic maculopathies like plaquenil maculopathy, spherical equivalent of the refractive error demonstrating >6 dioptres of myopia or an axial length of >26 mm, evidence or history of exudative (wet) AMD including evidence of retinal pigment epithelium (RPE) rips or evidence of neovascularization anywhere in the retina based on fluorescein angiogram in either eye within 12 months, retinal disease likely to confound visual performance or be affected by intraocular steroid, Intraocular surgery (including lens replacement surgery) within 3 months prior to dosing, aphakia or absence of the posterior capsule, previous violation of the posterior capsule is also excluded unless it occurred as a result of yttrium aluminum garnet (YAG) laser posterior capsulotomy in association with prior posterior chamber intraocular lens implantation and at least 60 days prior to Day 0, glaucoma or family history of glaucoma, any contraindication of IVT injection including current ocular or periocular infection, history of uveitis or endophthalmitis, history of IVT injection within 12 months.

If both eyes met the criteria, the eye with the best visual acuity at the screening visit was designated as the study eye.

### Study Protocol

A full ophthalmic assessment was performed at each study visit which included GA area assessed through FAF imaging, BCVA, lower-luminance BCVA (LL-BCVA) and intra-ocular pressure (IOP). Participants were assessed at screening, baseline, day 1, day 7, day 14, day 28, day 60, day 90 and day 150 (end of study). Blood and urine samples were collected for safety analysis at screening, baseline, day 7, day 28 and day 150.

Part 1 of the study involved a single participant treated with 1 mg/0.1 ml FCA to assess safety and tolerability. This participant was followed-up for 28 days before the results were reviewed by an independent data safety monitoring committee (DSMB). Subsequent to approval by the DSMB, a further 2 participants were treated with 1 mg/0.1 ml FCA and followed-up for a further 28 days after treatment before commencement of part 2.

Part 2 involved a single dose of 2 mg/0.1 ml of FCA in a single participant and followed-up for 28 days like part 1. The DSMB reviewed the results prior to enrolment of the remaining 5 participants.

### Outcome Measures

Fundus auto-fluorescence was captured using Heidelberg Spectralis (Heidelberg Engineering, Heidelberg, Germany). The assessment of GA size and progression was performed using FAF was performed by 2 graders (T.H and E.C.) in a blinded manner using Heidelberg region finder software version 2.6.2.0 (FIG. 1), a semi-automated program used to quantify atrophic areas. Baseline FAF images were defined and used to assess subsequent visits using the region finder software. In cases where there was a discrepancy of more than 20% between the two graders, a third grader (A.C.) evaluated the images. Areas of peri-papillary atrophy were not included in the measurements. GA was defined as well-demarcated regions of hypo-fluorescence on FAF from the absence of the RPE layer over the neurosensory retina (Refs).

BCVA was assessed at every study visit at 4 meters using an ETDRS chart following subjective refraction. LL-BCVA was assessed similarly with a neutral density lens. IOP was assessed with Goldmann applanation tonometry. Adverse events were reported from the first dose of FCA in the first patient until the last patient last visit.

### Statistical Analysis

Statistical analysis was performed using SPSS software version 24.0 (SPSS, Inc, Chicago, IL) and are primarily descriptive. Summaries of safety data are presented in the results. Descriptive statistics (mean, standard deviation (SD), median, minimum and maximum) are calculated for summaries of continuous data. Paired t-tests were performed to assess change from baseline and two sampled t-tests were performed to compare change between eyes.

Safety data, including vital signs, clinical safety labs and adverse events, will be summarised. Adverse events (AEs) were coded using the Medical Dictionary for Regulatory Activities (MedDRA), and data will be summarised by System Organ Class and preferred term.

### Results

Nine participants were enrolled in the study, baseline characteristics are presented in Table 3. The mean age of participants was 79.7±6.2 years and 55% were female. Mean baseline BCVA and LL-BCVA were 53.1±10.0 and 39.3±11.2 letters respectively. Mean baseline area of GA was 9.50±5.7.

TABLE 3
Baseline Characteristics

| Baseline Characteristic | N = 9 |
|---|---|
| Age (years) Mean ± SD | 79.73 ± 6.19 |
| Female | 55.5% |
| Right Eye | 55.5% |
| Pseudophakic | 55.5% |
| BCVA (letters) Mean ± SD | 53.11 ± 10.01 |
| LLVA (letters) Mean ± SD | 39.33 ± 11.21 |
| IOP Mean ± SD | 13.33 ± 3.08 |
| GA area (mm²) Mean ± SD | 9.53 ± 5.69 |

The initial pilot participant dosed with 1 mg/ml fludrocortisone acetate experienced a loss of 22 letters (≥15 letters) at day 14, however this was deemed most likely unrelated to the IP by the DSMB as there were no signs of any other adverse events or safety concerns.

One participant dosed with 2 mg/ml fludrocortisone acetate experienced a nasal subconjunctival haemorrhage after day 90 in the study eye that resolved after 1 week. No other adverse events were noted throughout the study.

No significant increases in IOP (≥10 mmHg) were observed among any patients throughout the study. The mean change in IOP was −0.25 mmHg (p=0.75) at day 150 compared to baseline Table 4. There were no significant changes in body temperature, heart rate, systolic or diastolic blood pressure throughout the study (p>0.05 for all).

Four of the 9 patients (44.4%) had a natural lens; no formal grading of lens opacity was performed as part of the protocol. One participant had significant lens opacity at screening which did not hinder imaging, however no participants required/underwent cataract surgery during the study period.

TABLE 4
Changes in characteristics from Baseline

| | Study Eye | | Fellow Eye | | P value |
|---|---|---|---|---|---|
| | Mean Change | | Mean Change | | between |
| Variable | from Baseline | P value | from Baseline | P value | eyes |
| BCVA (letters) | −2.63 ± 7.01 | 0.28 | 4.88 ± 8.37 | 0.69 | 0.07 |
| LLVA (letters) | 3.25 ± 9.23 | 0.38 | 4.5 ± 11.49 | 0.72 | 0.81 |
| IOP (mmHg) | −0.25 ± 4.06 | 0.75 | −0.625 ± 4.24 | 0.69 | 0.86 |
| GA area (mm²) | 0.5 ± 5.69 | 0.003 | 0.62 ± 4.49 | 0.02 | 0.64 |

### Progression of GA Area

The mean area of geographic atrophy increased in both the study (0.5 mm2 p=0.003) and fellow (0.62 mm2 p=0.02) eyes over the duration of the study. The change in area was not significant between eyes (p=0.64) Table 4.

### DISCUSSION

This study shows that intravitreal FCA is clinically safe and well-tolerated among this cohort of patients with geographic atrophy secondary to AMD. Typical side effects of intraocular steroids including raised intraocular pressure and lens opacity was not observed in this study. Furthermore, no systemic adverse event was observed during this does escalation study.

A recent meta-analysis of 23 studies reported the natural progression of GA lesions in untreated eyes to be 1.66 mm2/year (16). Results from the Proxima A and B clinical trials reported a growth rate of 2.09 and 1.90 mm2/year respectively over the first 12 months. Another study by Schmitz-Valckenberg et al (17) reported a change of 0.88 mm2 over 6 months. Our study findings indicate that GA progression was lower in both the treated (0.5 mm2) and fellow eyes (0.62 mm2) over the study period which may indicate a possible treatment effect of FCA.

Low luminance visual acuity is known to be a predictor of visual loss in patients with GA, where it can detect a change in central function earlier than standard VA assessments (18-20). Our findings show that LLVA did not deteriorate over the study period and may be considered promising, and worthy of a more detailed study to be conducted in a larger cohort.

Previous studies assessing intravitreal glucocorticoid treatments have found improvements in the function of the blood retinal barrier (BRB) (21, 22). These findings are consistent with pre-clinical investigations into the use of FCA in vitro, and in vivo using a mouse model of AMD (23). In those experiments FCA was found to have potent anti-inflammatory effects and to be neuroprotective in the AMD model.

Clinical studies have previously reported previously that monocular treatments can affect fellow eyes (24-26), meaning that comparison of measures between treated and fellow eyes cannot serve as valid controls, and that population norms are the most useful comparators. Given that both the treated and fellow eyes showed a slower progression rate compared to previous report, there may also be a similar sympathetic response in the fellow untreated eye.

Maddess et al has reported recently that there is a significant correlation between peripheral macular areas of treated and untreated nAMD eyes treated with anti-VEGF, based on monthly assessments for 15 months (ref Rai et al., in press). We suggest therefore that the findings may indicate that intraocular injections of FCA at 2 mg/ml may reduce the rate of GA progression, although a much larger study group followed over a longer timeframe is required to formally make such a conclusion.

The limitations of this study are the small sample size and the absence of formal lens grading, both of which were considered non-essential to the primary objective of this safety study. Another limitation of the study includes the small sample size and short duration of the study. Strengths of this study includes its prospective design.

In summary, the data show that intravitreal FCA is clinically safe and well-tolerated. Treatment with FCA did not increase IOP and has promising indications in terms of GA lesion growth and LLVA. Further longer-term studies with larger sample size and multi-dose regimes may aid in assessing the efficacy of FCA in reducing GA progression.

While this invention has been described in connection with specific embodiments thereof, it will be understood that it is capable of further modification(s). This application is intended to cover any variations uses or adaptations of the invention following in general, the principles of the invention and including such departures from the present disclosure as come within known or customary practice within the art to which the invention pertains and as may be applied to the essential features hereinbefore set forth.

As the present invention may be embodied in several forms without departing from the spirit of the essential characteristics of the invention, it should be understood that the above described embodiments are not to limit the present invention unless otherwise specified, but rather should be construed broadly within the spirit and scope of the invention as defined in the appended claims. The described embodiments are to be considered in all respects as illustrative only and not restrictive.

Various modifications and equivalent arrangements are intended to be included within the spirit and scope of the invention and appended claims. Therefore, the specific embodiments are to be understood to be illustrative of the many ways in which the principles of the present invention may be practiced. In the following claims, means-plus-function clauses are intended to cover structures as performing the defined function and not only structural equivalents, but also equivalent structures.

When a Markush group or other grouping is used herein, all individual members of the group and all combinations and sub-combinations possible of the group members are intended to be individually included in the disclosure. Every combination of components described or exemplified herein can be used to practice the invention, unless otherwise stated.

Whenever a range is given in the specification, for example, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. The broad term “comprising” is intended to encompass the narrower “consisting essentially of” and the even narrower “consisting of”. Thus, in any recitation herein of a phrase “comprising one or more claim element” (e.g., “comprising A), the phrase is intended to encompass the narrower, for example, “consisting essentially of A” and “consisting of A” Thus, the broader word “comprising” is intended to provide specific support in each use herein for either “consisting essentially of” or “consisting of”. The invention illustratively described herein suitably may be practiced in the absence of any element or elements, limitation or limitations which is not specifically disclosed herein.

One of ordinary skill in the art will appreciate that materials and methods, other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by examples, preferred embodiments and optional features, modification and variation of the concepts herein disclosed may be resorted to by those skilled in the art, and that such modifications and variations are considered to be within the scope of this invention as defined by the appended claims.

Each reference cited herein is incorporated in its entirety. Such references may provide sources of materials; alternative materials, details of methods, as well as additional uses of the invention.

### REFERENCES

1. Mitchell P, Smith W, Attebo K, Wang J J. Prevalence of age-related maculopathy in Australia. The Blue Mountains Eye Study. Ophthalmology. 1995; 102(10):1450-60.
2. Klein R, Klein B E, Linton K L. Prevalence of age-related maculopathy. The Beaver Dam Eye Study. Ophthalmology. 1992; 99(6):933-43.
3. Klein R, Klein B E, Tomany S C, Moss S E. Ten-year incidence of age-related maculopathy and smoking and drinking: the Beaver Dam Eye Study. American journal of epidemiology. 2002; 156(7):589-98.
4. Michalska-Malecka K, Kabiesz A, Nowak M, Spiewak D. Age related macular degeneration—challenge for future: Pathogenesis and new perspectives for the treatment. European Geriatric Medicine. 2015; 6(1):69-75.
5. Holz F G, Strauss E C, Schmitz-Valckenberg S, van Lookeren Campagne M. Geographic atrophy: clinical features and potential therapeutic approaches. Ophthalmology. 2014; 121(5):1079-91.
6. Coleman H R, Chan C C, Ferris F L, 3rd, Chew E Y. Age-related macular degeneration. Lancet (London, England). 2008; 372(9652):1835-45.
7. Klein R, Klein B E, Knudtson M D, Meuer S M, Swift M, Gangnon R E. Fifteen-year cumulative incidence of age-related macular degeneration: the Beaver Dam Eye Study. Ophthalmology. 2007; 114(2):253-62.
8. Klein R, Cruickshanks K J, Nash S D, Krantz E M, Nieto F J, Huang G H, et al. The prevalence of age-related macular degeneration and associated risk factors. Archives of ophthalmology (Chicago, 111: 1960). 2010; 128(6):750-8.
9. Chong E W, Wong T Y, Kreis A J, Simpson J A, Guymer R H. Dietary antioxidants and primary prevention of age related macular degeneration: systematic review and meta-analysis. BMJ (Clinical research ed). 2007; 335(7623):755.
10. Ding X, Patel M, Chan C C. Molecular pathology of age-related macular degeneration. Progress in retinal and eye research. 2009; 28(1):1-18.
11. Dastgheib K, Green W R. Granulomatous reaction to Bruch's membrane in age-related macular degeneration. Archives of ophthalmology (Chicago, 111: 1960). 1994; 112(6):813-8.
12. Penfold P L, Wong J G, Gyory J, Billson F A. Effects of triamcinolone acetonide on microglial morphology and quantitative expression of MHC-II in exudative age-related macular degeneration. Clinical & experimental ophthalmology. 2001; 29(3):188-92.
13. Krogh Nielsen M, Subhi Y, Molbech C R, Falk M K, Nissen M H, Sørensen T L. Systemic Levels of Interleukin-6 Correlate With Progression Rate of Geographic Atrophy Secondary to Age-Related Macular Degeneration. Invest Ophthalmol Vis Sci. 2019; 60(1):202-8.
14. Gomez-Sanchez C E, Kirk D N, Farrant R D, Milewich L. 18-substituted steroids: Synthesis of 18-hydroxycortisol (11β,17α,18,21-tetrahydroxy-4-pregnene-3,20-dione) and 18-hydroxycortisone (17α, 18,21-trihydroxy-4-pregnene-3,11,20-trione). Journal of Steroid Biochemistry. 1985; 22(1):141-6.
15. Tao Y, Jonas J B. Intravitreal triamcinolone. Ophthalmologica. 2011; 225(1):1-20.
16. Wang J, Ying G S. Growth Rate of Geographic Atrophy Secondary to Age-Related Macular Degeneration: A Meta-Analysis of Natural History Studies and Implications for Designing Future Trials. Ophthalmic Res. 2021; 64(2):205-15.
17. Schmitz-Valckenberg S, Sahel J A, Danis R, Fleckenstein M, Jaffe G J, Wolf S, et al. Natural History of Geographic Atrophy Progression Secondary to Age-Related Macular Degeneration (Geographic Atrophy Progression Study). Ophthalmology. 2016; 123(2):361-8.
18. Wood L J, Jolly J K, Buckley T M, Josan A S, MacLaren R E. Low luminance visual acuity as a clinical measure and clinical trial outcome measure: a scoping review. Ophthalmic Physiol Opt. 2021; 41(2):213-23.
19. Sunness J S, Rubin G S, Broman A, Applegate C A, Bressler N M, Hawkins B S. Low luminance visual dysfunction as a predictor of subsequent visual acuity loss from geographic atrophy in age-related macular degeneration. Ophthalmology. 2008; 115(9):1480-8, 8.e1-2.
20. Wu Z, Ayton L N, Luu C D, Guymer R H. Longitudinal changes in microperimetry and low luminance visual acuity in age-related macular degeneration. JAMA Ophthalmol. 2015; 133(4):442-8.
21. van der Wijk A E, Canning P, van Heijningen R P, Vogels I M C, van Noorden C J F, Klaassen I, et al. Glucocorticoids exert differential effects on the endothelium in an in vitro model of the blood-retinal barrier. Acta Ophthalmol. 2019; 97(2):214-24.
22. Lee J Y, Eun C K, Kim Y W, Kim H J, Jung Y J, Jae S Y, et al. The steroid effect on the blood-ocular barrier change induced by triolein emulsion as seen on contrast-enhanced MR images. Korean J Radiol. 2008; 9(3):205-11.
23. Racic T, Chang A, Fernando N, Brandli A, Natoli R, Penfold P, et al. Anti-inflammatory and neuroprotective properties of the corticosteroid fludrocortisone in retinal degeneration. Exp Eye Res. 2021; 212:108765.
24. Aghayeva F A, Chronopoulos P, Schuster A K, Pfeiffer N, Hoffmann E M. Inter-eye relationship of intraocular pressure change after unilateral trabeculectomy, filtering canaloplasty, or PreserFlo™ microshunt implantation. Graefes Arch Clin Exp Ophthalmol. 2021; 259(10):3045-53.
25. Gibbens M V. Sympathetic influences on the consensual ophthalmotonic reaction. Br J Ophthalmol. 1988; 72(10):750-3.
26. Rao H L, Senthil S, Garudadri C S. Contralateral intraocular pressure lowering effect of prostaglandin analogues. Indian J Ophthalmol. 2014; 62(5):575-9.

1: A method for treating a subject having a retinal eye condition that is refractory to anti-VEGF treatments, the method comprising administering to the subject a therapeutically effective amount of one or more steroids subsequent to anti-VEGF treatment, thereby treating the retinal eye condition.

2: A method for treating a subject having a retinal eye condition that is refractory to anti-VEGF treatments, the method comprising administering to the subject a therapeutically effective amount of one or more compounds capable of modulating an activity of a steroid receptor, prefer-ably a glucocorticoid receptor and/or mineralocorticoid receptor, subsequent to anti-VEGF treatment, thereby treating the retinal eye condition.

3: A method for treating a subject having a retinal eye condition that is refractory to anti-VEGF treatments, the method comprising administering to the individual:
(a) a therapeutically acceptable formulation of a steroid suitable for delivery to the eye, and
(b) at least a second therapeutically active compound in a concentration and dose sufficient to ameliorate the retinal eye condition, subsequent to anti-VEGF treatment.

4: A method according to claim 1, wherein the steroid is one or more mineralocorticoid or glucocorticoid or a therapeutically active analogue, derivative, homolog, pharmaceutically acceptable salt or conjugate thereof.

5: A method according to claim 4, wherein the one or more mineralocorticoid and/or more glucocorticoid or a therapeutically active analogue, derivative, homolog, pharmaceutically acceptable salt or conjugate thereof comprises one or more dual action compounds, wherein each dual action compound is capable of modulating the activity of both a mineralocorticoid receptor and a glucocorticoid receptor.

6: The method according to claim 4, wherein the one or more mineralocorticoid or a therapeutically active analogue, derivative, homolog, pharmaceutically acceptable salt or conjugate thereof comprises one or more of: 11-desoxycortisone (11-DC); fludrocortisone; fludrocortisone acetate (FA); fludrocortisone acetonide; Deoxycorticosterone acetate (DA); Deoxycorticosterone (DS); or Aldosterone; or a therapeutically active analogue, derivative, homolog, pharmaceutically acceptable salt or conjugate thereof.

7: The method according to claim 4, wherein the one or more glucocorticoid or a therapeutically active analogue, derivative, homolog, pharmaceutically acceptable salt or conjugate thereof may comprise one or more of: cortisol, cortisone, prednisone, prednisolone, methylprednisolone, dexamethasone, betamethasone, triamcinolone, triamcinolone acetonide, beclomethasone, fluocinolone or a therapeutically active analogue, derivative, homolog, pharmaceutically acceptable salt or conjugate thereof.

8: The method according to claim 51 wherein the dual action compound comprises one or more of triamcinolone; triamcinolone acetonide; cortisol; cortisone; prednisone; prednisolone; methylprednisolone; fludrocortisone; fludrocortisone acetate; fludrocortisone acetonide; or a therapeutically active analogue, derivative, homolog, pharmaceutically acceptable salt or conjugate thereof.

9: The method according to claim 4, wherein the one or more mineralocorticoid or one or more glucocorticoid or a therapeutically active analogue, derivative, homolog, pharmaceutically acceptable salt or conjugate thereof comprises fludrocortisone or a therapeutically active analogue, derivative, homolog, pharmaceutically acceptable salt or conjugate thereof, optionally wherein the therapeutically active analogue, derivative, homolog, pharmaceutically acceptable salt or conjugate thereof may comprise one or more of fludrocortisone acetate and fludrocortisone acetonide.

10: The method according to claim 4, wherein the one or more mineralocorticoid and/or one or more glucocorticoid or a therapeutically active analogue, derivative, homolog, pharmaceutically acceptable salt or conjugate thereof comprises triamcinolone acetonide or a therapeutically active analogue, derivative, homolog, pharmaceutically acceptable salt or conjugate thereof.

11: The method according to claim 1, wherein the retinal eye condition is macular edema (ME), diabetic macular edema (DME), or age related macular degeneration (AMD) including wet-AMD or dry AMD.

12: The method according to claim 1, wherein the retinal eye condition comprises: an exudative eye condition, a back of the eye condition, macular degeneration including age-related macular degeneration (AMD) including both the dry (geographic atrophy) and wet (choroidal neovascularisation (CNV)), macula edema (ME) including diabetic macular edema (DME), angio-graphic cystoid macular edema, cistoid macular edema (CMO), diabetic retinopathy, (DR) including proliferative diabetic retinopathy (PDR) and retinal vein occlusion including central retinal vein occlusion (CRVO) or branch retinal vein occlusion (BRVO) maculopathy including an age related maculopathy (ARM), an exudative eye disease or condition, retinal pigment epithelium detachments (PED), forms of age related macular degeneration, a diabetic eye disease or condition including a diabetic retinopathy, corneal neovascularisation, cyclitis, Hippel-Lindel disease, retinopathy of prematurity or retrolental fibroplasia, pterygium, histoplasmosis, iris neovascularisation, glaucoma, glaucoma-associated neovascularisation, Purtcher's retinopathy, ocular hypertension, macular oedema, Coats' disease, uveitis including anterior uveitis, Sicca syndrome, hereditary diseases associated with increased extra-intracellular lipid storage/accumulation, juvenile macular degeneration, an ocular allergy and an ocular tumour.

13: The method according to claim 12, wherein the eye disease or condition comprises a back of eye disease or condition, comprising an exudative back of eye exudative disease or condition, optionally the back of eye disease or condition comprises an eye disease or condition involving the retina, macular and/or fovea in the posterior region of the eye.

14: The method according to claim 1, wherein the eye disease or condition comprises dry AMD, optionally early AMD and geographic atrophy (GA), distinct from exudative AMD.

15: The method according to claim 1, wherein the eye disease and/or condition is a diabetic eye disease and/or condition.

16: The method according to claim 1, wherein the therapeutically effective amount comprises a range of about 0.01 wt % to about 10 wt %, optionally about 0.025 wt % to about 2.5 wt %.

17: The method according to claim 1, wherein the one or more steroids are injected into the eye, optionally the injection comprises a suprachoroidal injection.

18: The method according to claim 1, wherein the one or more steroids are provided in a unit-dose formulation, optionally the unit dose formulation is provided in a pre-filled syringe, optionally the pre-filled syringe comprises two barrels.

19: The method according to claim 1, wherein one or more pharmaceutically acceptable carriers, diluents or excipients are comprised, one or more surfactant or wetting agent.

20: The method according to claim 19, wherein the one or more pharmaceutically acceptable carrier comprises hemp, hemp oil or a pharmaceutically effective hemp or hemp oil extract, optionally the carrier comprises hemp oil comprising about 80% to 90% balanced Omega fatty acids.

21: The method according to claim 20, wherein the hemp, hemp oil or a pharmaceutically effective extract is for use or when used as a carrier or delivery vehicle for the one or more steroids.
